(19) United States
(12) Patent Application Publication
(10) Pub. No.: US 2023/0094301 A1
(43) Pub. Date: Mar. 30, 2023

(54) DETERMINING TRANSIENT DECELERATIONS

(71) Applicant: Covidien LP, Mansfield, MA (US)

(72) Inventors: Pedro M. Elias Monteiro Gomes, Sobral de Monte Agraco (PT); Scott McGonigle, Loanhead (GB); Dean Montgomery, Edinburgh (GB); Paul S. Addison, Edinburgh (GB)

(21) Appl. No.: 17/822,269

(22) Filed: Aug. 25, 2022

Related U.S. Application Data
(60) Provisional application No. 63/248,857, filed on Sep. 27, 2021.

(51) Int. Cl. A61B 5/0205; A61B 5/145; A61B 5/00

(52) U.S. Cl. CPC A61B 5/0205; A61B 5/14542; A61B 5/7289; A61B 5/7278; A61B 5/02405

(57) ABSTRACT

An example device for determining one or more transient decelerations includes a memory configured to store a sensed pulse rate signal indicative of one or more sensed pulse rates and processing circuitry. The processing circuitry is configured to determine that an amplitude threshold is crossed by a sensed pulse rate signal indicative of one or more sensed pulse rates. The processing circuitry also is configured to, from a time the amplitude threshold is crossed, determine that a pulse rate returns to within a range of a baseline pulse rate within a number of samples or a time period. The processing circuitry is also configured to, based on the pulse rate returning to within the range of the baseline pulse rate, from the time the amplitude threshold is crossed, within the number of samples or the time period, determine a transient deceleration.

This application claims priority to U.S. Provisional Application No. 63/248,857, filed Sep. 27, 2021, the entire contents of which is hereby incorporated by reference.

### TECHNICAL FIELD

This disclosure relates to systems, devices, and methods for patient monitoring.

### BACKGROUND

Oximetry may be used in the clinical setting to measure, in a non-invasive manner, blood characteristics. For example, oximetry may be used to estimate arterial blood oxygenation and sense the pulse rate of a patient, which may be a proxy for heart rate. To sense pulse rate, two optical sources, typically light-emitting-diodes (LEDs), are used to inject light into tissue of the patient. A photodiode may be used to capture the light after propagating through blood perfused tissue. During a cardiac cycle, the amount of blood in the optical path changes which changes the amount the light that is absorbed by the photodiode. As more light is absorbed, the photodiode produces less photocurrent. Hence, during the cardiac cycle the photocurrent from the photodiode is a modulated photocurrent producing a pulsatile waveform associated with each heart beat. The period between each beat is the pulse period. The pulse rate may be derived from this pulsatile waveform.

### SUMMARY

In general, this disclosure relates to devices, systems, and techniques for detecting transient decelerations in pulse rate signals. HRV provides an estimation of the amount of variability in heart rate. In some examples, HRV can be calculated as the standard deviation of the pulse rate (used as proxy for heart rate) over a defined time period.

Among other fluctuations displayed in a pulse rate, the detection of specific cases of very sharp decelerations in pulse rate followed by an equally quick return may be of interest. This disclosure describes example techniques for detection of such sharp decelerations. In some cases, detection of very sharp decelerations in pulse rate, such as neonatal pulse rate, may be for clinical usage and research in neonatal intensive care unit (NICU) settings. Such deceleration events have been associated with late-onset clinical sepsis. However, fluctuations displayed in the pulse rate may be of interested in other settings and for other purposes as well, and the techniques should not be considered limited to neonatal pulse rate or for late-onset clinical sepsis.

In one example, a device for determining a transient deceleration includes: a memory configured to store a sensed pulse rate signal indicative of one or more sensed pulse rates; and processing circuitry configured to: determine that an amplitude threshold is crossed by the sensed pulse rate signal; from a time the amplitude threshold is crossed, determine that a pulse rate returns to within a range of a baseline pulse rate within a number of samples or a time period; based on the pulse rate returning to within the range of the baseline pulse rate, from the time the amplitude threshold is crossed, within the number of samples or the time period, determine a transient deceleration; and output information indicative of the determined transient deceleration.

In another example, a method for determining a transient deceleration includes: determining, by processing circuitry, that an amplitude threshold is crossed by a sensed pulse rate signal indicative of one or more sensed pulse rates; from a time the amplitude threshold is crossed, determining, by the processing circuitry, that a pulse rate returns to within a range of a baseline pulse rate within a number of samples or a time period; based on the pulse rate returning to within the range of the baseline pulse rate, from the time the amplitude threshold is crossed, within the number of samples or the time period, determining, by the processing circuitry, a transient deceleration; and outputting, by the processing circuitry, information indicative of the determined transient deceleration.

In one example, a non-transitory computer-readable medium includes instructions, which, when executed, cause processing circuitry to: determine that an amplitude threshold is crossed by the sensed pulse rate signal; from a time the amplitude threshold is crossed, determine that a pulse rate returns to within a range of a baseline pulse rate within a number of samples or a time period; based on the pulse rate returning to within the range of the baseline pulse rate, from the time the amplitude threshold is crossed, within the number of samples or the time period, determine a transient deceleration; and output information indicative of the determined transient deceleration.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1 is a conceptual block diagram illustrating an example oximetry device.

FIGS. 2A and 2B are graphical diagrams illustrating an example of non-transient decelerations and a transient deceleration, respectively.

FIG. 3 is a conceptual block diagram illustrating an example oximetry device configured to monitor heart rate of a patient.

FIGS. 4A and 4B are graphical diagrams illustrating example changes in pulse rate and associated skews to HRV.

FIG. 5 is a graphical diagram depicting an example classification of a transient deceleration using thresholds.

FIG. 6 is a flow diagram illustrating an example technique for determining a transient deceleration.

### DETAILED DESCRIPTION

In some cases, a pulse rate signal, such as a neonatal pulse rate signal but not limited to neonatal pulse rate signal, may include a sudden and relatively large reduction in pulse rate followed by a return to an initial baseline pulse rate, which may be referred to as a “transient deceleration.” The appearance of transient decelerations in pulse rate signals or heart rate signals has been connected to poor clinical outcomes, such as sepsis. See, for example, Fairchild K D, O'Shea T M. Heart Rate Characteristics: Physiomarkers for Detection of Late-Onset Neonatal Sepsis. Clin Perinatol. 2010; 37(3):581-598. doi:10.1016/j.clp.2010.06.002; and Joshi R, Kommers D, Oosterwijk L, Feijs L, Van Pul C, Andriessen P. Predicting Neonatal Sepsis Using Features of Heart Rate Variability, Respiratory Characteristics, and ECG-Derived Estimates of Infant Motion. IEEE J Biomed Heal Informatics. 2020; 24(3):681-692. doi:10.1109/JBHI.2019.2927463.

Therefore, it may be desirable to have a system, device, or method capable of distinguishing transient decelerations from other types of fluctuations in a pulse rate or heart rate signal and capable of detecting, counting, and characterizing the transient decelerations.

A reduction in heart rate variability (HRV) has been connected to clinical outcomes such as sepsis and intraventricular hemorrhage, among others. However, transient decelerations have a substantial impact on HRV values leading to an increase in the calculated value of HRV (e.g., in a moving 5-minute standard deviation). As such, transient decelerations act as a confounder to the use of HRV values to detect the deterioration of a patient. Therefore, the detection, count and/or removal of transient decelerations from the HRV calculation may improve the clinical use of HRV values, and may also provide additional useful information to the clinician. Because the HRV values may be calculated based on a 5-minute standard deviation, any error caused by a transient deviation to the HRV values may propagate for that time period.

An oximeter is a medical device configured to determine an oxygen saturation of an analyzed tissue. An oximeter may also be configured to sense a pulse rate of a person, such as a patient. An oximeter may measure other characteristics and chemical compositions of blood, like carbon monoxide.

An oximeter may include a sensor device that is placed at a site on a patient, for example, on a fingertip, toe, forehead or earlobe, the cerebral cortex, or in the case of a neonate, across a foot, across a hand, or another location. The oximeter may use a light source to pass light through blood perfused tissue and photoelectrically sense the absorption of the light in the tissue. Additional suitable sensor locations may include, for example, a neck to monitor carotid artery pulsatile flow, a wrist to monitor radial artery pulsatile flow, an inside of a patient's thigh to monitor femoral artery pulsatile flow, an ankle to monitor tibial artery pulsatile flow, around or in front of an ear, locations with strong pulsatile arterial flow, or other locations.

The oximeter may be configured to output a photonic signal that interacts with tissue at one or more wavelengths that are attenuated by the blood in an amount representative of the blood constituent concentration. The oximeter may be configured to generate the photonic signal at red and infrared (IR) wavelengths. The oximeter may estimate the blood oxygen saturation of hemoglobin in arterial blood based on an intensity of the photonic signal at the red wavelength and the photonic signal at the infrared wavelength.

Light emitting diodes (LEDs) of an oximeter may be manufactured to output a photonic signal at a particular wavelength with a manufacturing tolerance. For example, a first LED may output a first phonic signal (e.g., red light) at a first wavelength range (e.g., 630 nm-700 nm) with a first manufacturing tolerance of 5%. In this example, a second LED may output a second phonic signal (e.g., infrared light) at a second wavelength range (e.g., 700 nm-1200 nm) with a second manufacturing tolerance of 5%. While various examples described herein refer to a LED that may output relatively low intensity light, in some examples, LEDs may include devices that output relatively intense beams of light of infrared radiation (e.g., laser diodes), vertical-cavity surface-emitting laser, or another device that emits light using at least one p-type junction and at least one n-type junction. Moreover, while examples described herein may refer to a device emitting light (e.g., LED, laser diode, etc.) similar techniques may be used with devices that receive light (e.g., photodiodes).

In accordance with the techniques of the disclosure, a device (e.g., an oximeter) may be configured to determine a transient deceleration in a sensed pulse rate signal indicative of one or more sensed pulse rates. For example, a device may be configured to determine that an amplitude threshold is crossed by a sensed pulse rate signal. The device may be configured to, from a time the amplitude threshold is crossed, determine that a pulse rate returns to within a range of a baseline pulse rate within a number of samples or a time period. The device may be configured to, based on the pulse rate returning to within the range of the baseline pulse rate, from the time the amplitude threshold is crossed, within the number of samples or the time period, determine a transient deceleration. The device may be configured to output information indicative of the determined transient deceleration.

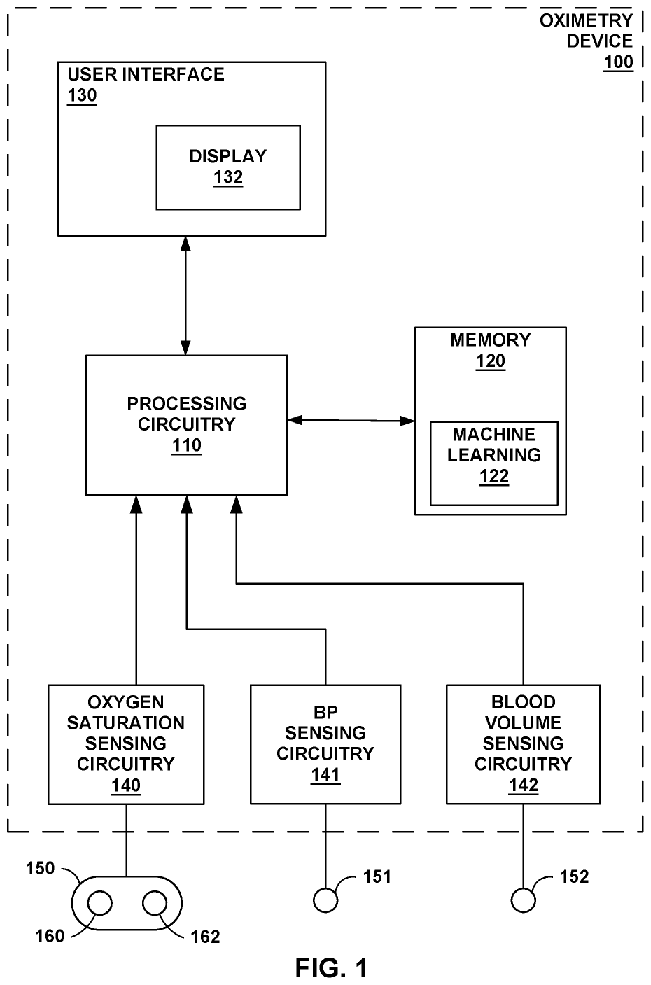

FIG. 1 is a conceptual block diagram illustrating an example oximetry device 100. While the example of FIG. 1 describes an oximetry device, techniques described herein for determining transient decelerations or HRV values may be used with other sources of pulse rate or heart rate, such as an electrocardiogram monitor or an arterial blood pressure monitor. The techniques herein may be applied to adult patients, child patients, neonatal patients, as well as healthy subjects.

Oximetry device 100 includes processing circuitry 110, memory 120, user interface 130, display 132, sensing circuitry 140, 141, and 142, and sensing device(s) 150, 151, and 152. In some examples, oximetry device 100 may be configured to determine transient decelerations e.g., during a medical procedure or for more long-term monitoring, such as monitoring of prenatal infants, children, or adults. A clinician may receive information regarding the transient decelerations of a patient via display 132 and adjust treatment or therapy to the patient based on the transient decelerations.

Processing circuitry 110 as well as other processors, processing circuitry, controllers, control circuitry, and the like, described herein, may include one or more processors. Processing circuitry 110 may include any combination of integrated circuitry, discrete logic circuitry, analog circuitry, such as one or more microprocessors, digital signal processors (DSPs), application specific integrated circuits (ASICs), or field-programmable gate arrays (FPGAs). In some examples, processing circuitry 110 may include multiple components, such as any combination of one or more microprocessors, one or more DSPs, one or more ASICs, or one or more FPGAs, as well as other discrete or integrated logic circuitry, and/or analog circuitry.

Memory 120 may be configured to store measurements of transient decelerations, HRV values, pulse rate, associated time stamps, blood pressure, oxygen saturation (e.g., SpO₂), blood volume, other physiological parameters, relationships between blood pressure and physiological parameters, mean arterial pressure (MAP) values, rSO2 values, COx values, BVS values, HVx values, for example. Memory 120 may also be configured to store data such as thresholds described herein or thresholds for detecting abrupt changes in blood pressure, previous LLA and ULA values, and/or other physiological parameters and expected values of physiological parameters. Memory 120 may also be configured to store data such as threshold levels for physiological parameters, threshold values for blood pressure, and/or threshold levels for signal quality metrics. The thresholds or other data may stay constant throughout the use of device 100 and across multiple patients, or these values may change over time.

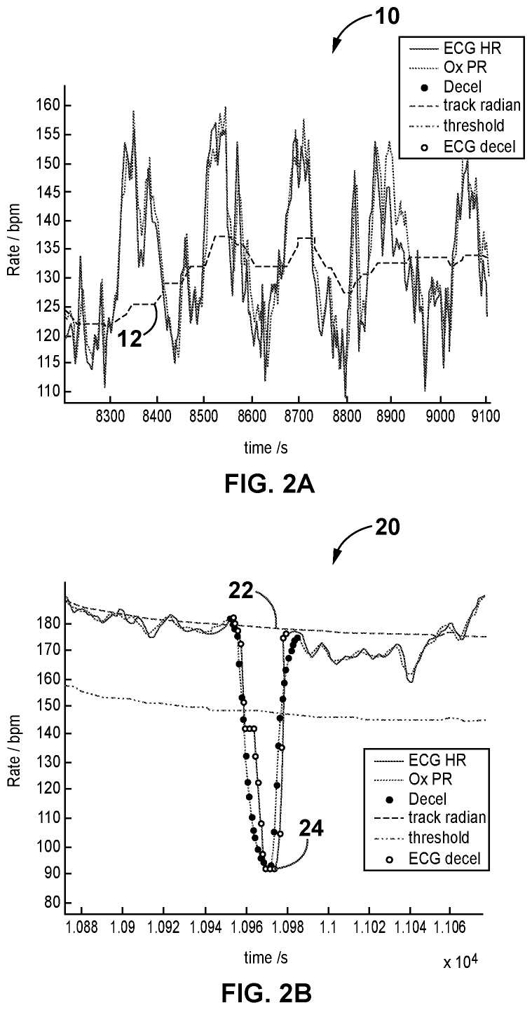

Memory 120 may store program instructions, which may include one or more program modules, which are executable by processing circuitry 110. When executed by processing circuitry 110, such program instructions may cause processing circuitry 110 to provide the functionality ascribed to it herein. For example, memory 120 may store instructions regarding how to determine transient decelerations in a sensed pulse rate (or heart rate) signal, abrupt changes in measured blood pressure, calculating ULA and LLA values, and presenting information to the user via user interface 130 or an external device. The program instructions may be embodied in software, and/or firmware. Memory 120, as well as other memory devices described herein (e.g., memory 220 shown in FIG. 2), may include any volatile, non-volatile, magnetic, optical, circuitry, or electrical media, such as a random access memory (RAM), read-only memory (ROM), non-volatile RAM (NVRAM), electrically-erasable programmable ROM (EEPROM), flash memory, or any other digital media.

User interface 130 and/or display 132 may be configured to present information to a user (e.g., a clinician). For example, display 132 may display transient decelerations, HRV values, pulse rate signals, skews, or the like. In some examples, display 132 may display profiles and/or statistics associated with transient decelerations, such as a count of transient decelerations, an average duration of transient decelerations, an average time period between transient decelerations, an average amplitude of transient decelerations, a count of transient decelerations per a time period (e.g., number of transient decelerations per hour), or the like. User interface 130 and/or display 132 may be configured to present a graphical user interface to a user, where each graphical user interface may include indications of values of one or more physiological parameters of a subject. For example, processing circuitry 110 may be configured to present transient decelerations, HRV values, blood pressure values, other physiological parameter values (e.g., pulse rate or heart rate) of a patient via display 132. As another example, processing circuitry 110 may present, via display 132, determined transient decelerations, HRV values, pulse rate information, respiration rate information, blood pressure, any other patient parameters, or any combination thereof.

User interface 130 and/or display 132 may include a monitor, cathode ray tube display, a flat panel display such as a liquid crystal (LCD) display, a plasma display, or a light emitting diode (LED) display, personal digital assistant, mobile phone, tablet computer, laptop computer, any other suitable display device, or any combination thereof. User interface 130 may also include means for projecting audio to a user, such as speaker(s). Processing circuitry 110 may be configured to present, via user interface 130, a visual, audible, or somatosensory notification (e.g., an alarm signal) indicative of a transient deceleration. User interface 130 may include or be part of any suitable device for conveying such information, including a computer workstation, a server, a desktop, a notebook, a laptop, a handheld computer, a mobile device, or the like. In some examples, processing circuitry 110 and user interface 130 may be part of the same device or supported within one housing (e.g., a computer or monitor).

Sensing circuitry 140, 141, and 142 may be configured to receive physiological signals sensed by respective sensing device(s) 150, 151, and 152 and communicate the physiological signals to processing circuitry 110. Sensing device(s) 150, 151, and 152 may include any sensing hardware configured to sense a physiological parameter of a patient, such as, but not limited to, one or more electrodes, optical receivers, blood pressure cuffs, or the like. Sensing circuitry 140, 141, and 142 may convert the physiological signals to usable signals for processing circuitry 110, such that processing circuitry 110 is configured to receive signals generated by sensing circuitry 140, 141, and 142. Sensing circuitry 140, 141, and 142 may receive signals indicating physiological parameters from a patient, such as, but not limited to, pulse rate, blood pressure, oxygen saturation, and respiration. Sensing circuitry 140, 141, and 142 may include, but are not limited to, blood pressure sensing circuitry, oxygen saturation sensing circuitry, pulse rate sensing circuitry, temperature sensing circuitry, electrocardiography (ECG) sensing circuitry, electroencephalogram (EEG) sensing circuitry, or any combination thereof. In some examples, sensing circuitry 140, 141, and 142 and/or processing circuitry 110 may include signal processing circuitry such as an analog-to-digital converter.

Oxygen saturation sensing device 150 is an oxygen saturation sensor configured to generate an oxygen saturation signal indicative of blood oxygen saturation within the venous, arterial, and/or capillary systems within a region of the patient. For example, oxygen saturation sensing device 150 may be configured to be placed on the patient's forehead and may be used to determine the oxygen saturation of the blood of the patient within the venous, arterial, and/or capillary systems of a region underlying the patient's forehead (e.g., in the cerebral cortex).

Oxygen saturation sensing device 150 may include emitter 160 and detector 162. Emitter 160 may include at least two light emitting diodes (LEDs), each configured to emit at different wavelengths of light, e.g., red and near infrared light. In some examples, light drive circuitry (e.g., within oxygen saturation sensing device 150, sensing circuitry 140, and/or processing circuitry 110) may provide a light drive signal to drive emitter 160 and to cause emitter 160 to emit light. In some examples, the LEDs of emitter 160 emit light in the wavelength range of about 600 nanometers (nm) to about 1000 nm. In a particular example, one LED of emitter 160 is configured to emit light at a wavelength of about 730 nm and the other LED of emitter 160 is configured to emit light at a wavelength of about 810 nm. Other wavelengths of light may also be used in other examples.

In some examples, detector 162 may include a single detection element. In other examples, a plurality of detection elements may be used. Light intensity of multiple wavelengths may be received at detector 162. For example, if two wavelengths are used, the two wavelengths may be contrasted to arrive at an oxygen saturation. Oxygen saturation sensing device 150 may provide the oxygen saturation signal to processing circuitry 110 or to any other suitable processing device.

Blood pressure sensing device 151 and oxygen saturation sensing device 150 may each be placed on the same or different parts of the patient's body. For example, blood pressure sensing device 151 and oxygen saturation sensing device 150 may be physically separate from each other and separately placed on the patient. As another example, blood pressure sensing device 151 and oxygen saturation sensing device 150 may in some cases be part of the same sensor or supported by a single sensor housing. For example, blood pressure sensing device 151 and oxygen saturation sensing device 150 may be part of an integrated oximetry system configured to non-invasively measure blood pressure (e.g., based on time delays in a PPG signal) and oxygen saturation. One or both of blood pressure sensing device 151 or oxygen saturation sensing device 150 may be further configured to measure other parameters, such as hemoglobin, respiratory rate, respiratory effort, pulse rate or heart rate, saturation pattern detection, response to stimulus such as bispectral index (BIS) or electromyography (EMG) response to electrical stimulus, or the like. While an example oximetry device 100 is shown in FIG. 1, the components illustrated in FIG. 1 are not intended to be limiting. Additional or alternative components and/or implementations may be used in other examples.

Blood pressure sensing device 151 may be any sensor or device configured to obtain the patient's blood pressure (e.g., arterial blood pressure). Blood pressure sensing device 151 may include a blood pressure cuff for non-invasively monitoring blood pressure or an arterial line for invasively monitoring blood pressure (e.g., a pressure probe configured to be placed within an artery or vein). In certain examples, blood pressure sensing device 151 may include one or more pulse oximetry sensors. In some such cases, the patient's blood pressure may be derived by processing time delays between two or more characteristic points within a single plethysmography (PPG) signal obtained from a single pulse oximetry sensor.

Processing circuitry 110 may be configured to receive one or more physiological signals generated by sensing devices 150, 151, and 152 and sensing circuitry 140, 141, and 142. The physiological signals may include a signal indicating pulse rate, a signal indicating blood pressure, a signal indicating oxygen saturation, and/or a signal indicating blood volume of a patient. Processing circuitry 110 may be configured to determine a relationship between blood pressure values of the patient and a physiological parameter of the patient, such as a correlation index (e.g., COx, a hemoglobin volume index (HVx)), an oxygen saturation value, a blood volume value, a gradient-based metric of two or more physiological parameters, and/or another physiological parameter. Processing circuitry 110 can determine a gradients-based metric by determining respective gradients of signals for physiological parameters and determining whether the respective gradients trend together.

In accordance with one or more techniques of this disclosure, processing circuitry 110 may be configured to determine transient decelerations in a sensed pulse rate signal. FIGS. 2A and 2B are graphical diagrams illustrating an example of non-transient decelerations and a transient deceleration, respectively. In one or more examples, a transient deceleration may be considered to be a reduction in pulse or heart rate from a reasonably well-defined baseline pulse rate where that drop exceeds an amplitude threshold (for example, a drop of 30 bpm, or a user-defined value), and where this drop in pulse rate is followed by an associated return to around the baseline pulse rate (e.g., an acceleration). In addition, this drop in the pulse rate and return to the baseline pulse rate occurs within a time period, for example, where the rate of change of heart rate is close to or greater than 1 bpm/s, and, for example, where this rate is calculated from both the initial baseline point to the deceleration minimum and from that minimum to a last point of the deceleration when returning to the baseline pulse rate. The transient deceleration event is based on a period of previously relatively stable baseline pulse rate where accelerations are absent. In the example of FIG. 2A, the valleys shown in waveform 10 are not considered transient decelerations. The baseline pulse rate 12 is around the midpoint between the pulse rate maximum and the pulse rate minimum of waveform 10. However, in the example of FIG. 2B, valley 24 of waveform 20 may be considered to be a transient deceleration as valley 24 starts around the baseline pulse rate 22 and returns to around baseline pulse rate 22.

This disclosure describes example techniques for processing circuitry 110 to determine whether a change in pulse rate is a transient deceleration or not. For example, with the example techniques described in this disclosure, processing circuitry 110 may be configured to accurately determine that the valleys shown in waveform 10 in FIG. 2A are not transient decelerations, and may be configured to accurately determine that valley 24 of waveform 20 in FIG. 2B is a transient deceleration. Based on the determination of whether a change in pulse rate is a transient deceleration or not, processing circuitry 110 may perform various example operations described in the disclosure to account for the transient deceleration, resulting in a more accurate HRV value.

For example, processing circuitry 110 may determine that an amplitude threshold is crossed by the sensed pulse rate signal. From a time the amplitude threshold is crossed, processing circuitry 110 may determine that a pulse rate returns to within a range of a baseline pulse rate within a number of samples or a time period. Based on the pulse rate returning to within the range of the baseline pulse rate, from the time the amplitude threshold is crossed, within the number of samples or the time period, processing circuitry 110 may determine a transient deceleration has occurred and output information indicative of the determined transient deceleration, for example, to display 132. In some examples, one or more of the amplitude threshold, the number of samples, or the time period may be predetermined, but example techniques of this disclosure are not limited to predetermined thresholds, samples, or time periods.

In some examples, processing circuitry 110 may monitor the sensed pulse rate signal to determine the one or more transient decelerations. In some examples, processing circuitry 110 may remove the one or more transient decelerations from the sensed pulse rate signal to create a modified pulse rate signal which processing circuitry 110 may store in memory 120 and/or output to display 132. Processing circuitry 110 may determine an HRV value based on the modified pulse rate signal and output information indicative of the determined HRV value, for example, to display 132. For example, processing circuitry 110 may determine a standard deviation in the modified pulse rate signal over a predetermined time period, such as 5 minutes. In this way, the transient decelerations may not impact the determination of the HRV.

In some examples, processing circuitry 110 may determine a baseline pulse rate. For example, processing circuitry 110 may use a lowpass filter on the sensed pulse rate signal to determine the baseline pulse rate. Processing circuitry 110 may determine a slope from an onset of a deceleration to a lower threshold. This lower threshold may be an amplitude threshold which can be user-defined, and may be referred to as a predetermined lower threshold, but the example techniques do not require the lower threshold to be predetermined. If the predetermined lower threshold is reached, processing circuitry 110 may count a number of samples from a timestamp associated with reaching of the predetermined lower threshold onward, until a sample or time limit is reached. The sample or time limit may be a predetermined sample or time limit, but the example techniques are not so limited.

Processing circuitry 110 may determine the deceleration to be a suspected transient deceleration based on the pulse rate returning to original baseline pulse rate, within a tolerance (e.g., predetermined tolerance), during this sample or time limit. In this way, processing circuitry 110 may initially determine suspected transient deceleration, and may then confirm whether a suspected transient deceleration actually correspond to a transient deceleration.

After the return to original baseline is detected, processing circuitry 110 may determine various metrics may be based on the suspected transient deceleration. For example, processing circuitry 110 may find an initial deceleration timestamp by searching backwards for the first peak before the predetermined lower threshold was crossed. This may ensure that the transient deceleration is detected from the origin of the transient deceleration. For example, processing circuitry 110 may check the amplitude from the initial transient deceleration (e.g., suspected transient deceleration) timestamp to a minimum value against the amplitude threshold to allow classification as a transient deceleration. In the case where processing circuitry 110 applies one or more lowpass filters to the sensed pulse rate signal, processing circuitry 110 may apply a data-trained calibration to the deceleration amplitude (e.g., multiplying factor of 1.2 or other data-trained value) to yield a more accurate deceleration amplitude. In some examples, processing circuitry 110 may output the deceleration amplitude value (e.g., a rate in bpm) inversely as a period in milliseconds, to provide a more familiar value to the clinician. Processing circuitry 110 may compare the period of the deceleration against the sample or time limit period to determine whether to classify the deceleration as a transient deceleration, as discussed above.

Processing circuitry 110 may compare the amplitude of the return pulse rate value to the original baseline value against the initial deceleration value to check for symmetry in the deceleration. For example, processing circuitry 110 may compare the amplitude of the return pulse rate value from the minimum of the deceleration to a ratio (e.g., a predetermined ratio) of the suspected deceleration amplitude. Processing circuitry 110 may then compare the ratio between the period and amplitude against a threshold (e.g., a predetermined threshold) to confirm the deceleration is an actual transient deceleration.

In some examples, processing circuitry 110 may calculate a median pulse rate over a sufficiently long period, e.g., 360 seconds, to determine the baseline pulse rate. In other examples, processing circuitry 110 may determine the baseline pulse rate using a lowpass filter, such as a lowpass filter of the type: signal_{lowpass}(t)=signal_{lowpass}(t−1)+A×(signal(t)−signal_{lowpass}(t−1)),

where A may be set to a value, such as 0.001, as an example.

In some examples, the amplitude threshold may be a percentage drop (e.g., 30%, 40% from the baseline pulse rate), or a drop in number of pulses (e.g., 30 bpm, 40 bpm, 50 bpm from the baseline pulse rate). In some examples, the amplitude threshold may be a drop to a predetermined value (e.g., if the rate drops below 100 bpm, 120 bpm, etc.).

In some examples, processing circuitry 110 may determine that the HRV of sensed pulse rate signal, immediately before the deceleration, is above a threshold (e.g., a predetermined threshold), for example, above 5 bpm. In such examples, processing circuitry 110 may disregard the deceleration (e.g., not remove the deceleration from the modified pulse rate signal or count the deceleration). This may be done in order to detect transient decelerations only during reduced HRV periods.

In some examples, processing circuitry 110 may determine the skewness of deceleration, for example, whether the skewness is in a single direction or more than one direction, or in which direction(s).

In another example, processing circuitry 110 may determine the HRV value with and without the decelerations included. For example, oximetry device 100 may determine a first HRV value based on the sensed pulse rate signal and a second HRV value based on the modified pulse rate signal. In some examples, processing circuitry 110 may store, display, and/or transmit information indicative of both the first HRV value and the second HRV value. In some examples, processing circuitry 110 may evaluate decelerations as the absolute or relative difference between the first HRV value and the second HRV value.

In some examples, processing circuitry 110 may use further qualifications to determine a transient deceleration (e.g., confirm that a suspected transient deceleration is an actual transient deceleration). Such further qualifications may include, but are not limited to, one or more of the following. For example, processing circuitry 110 may use template matching in which processing circuitry 110 may compare a predetermined and stored template of an archetypal transient deceleration to the sensed pulse rate signal and may associated large, localized correlations between the sensed pulse rate signal and the template with a transient deceleration event. In some examples, processing circuitry 110 may derive this archetypal transient deceleration template through ensemble averaging over a number of identified transient deceleration events (e.g., for different patients or the same patient) and store the archetypal transient deceleration template in memory. In some examples, processing circuitry 110 may employ a neural networks model or other machine learning or deep learning techniques, where labeled transient decelerations in the signal for many different patients or the same patient are used to train a model to detect transient deceleration events. For example, processing circuitry 110 may employ time frequency methods, including a wavelet transform, where the morphology of the time-frequency space may be interrogated to detect features associated with transient decelerations.

In some examples, processing circuitry 110 may omit blood pressure and/or blood volume circuitry. For example, processing circuitry 110 may omit sensing circuitry 141 and/or sensing device 151. In some examples, processing circuitry 110 may omit sensing circuitry 142 and sensing device 152. In some examples, processing circuitry 110 may include only circuitry for determining an oxygen saturation level and pulse rate.

In the above examples, processing circuitry 110 is described as performing the example techniques. However, any one or combination of processing circuitry 110, sensing circuitry 140, and/or sensing device 150 may be configured to perform the example techniques. For instance, the example techniques may be performed by circuitry, and example of the circuitry includes any one or any combination of processing circuitry 110, sensing circuitry 140, and/or sensing device 150.

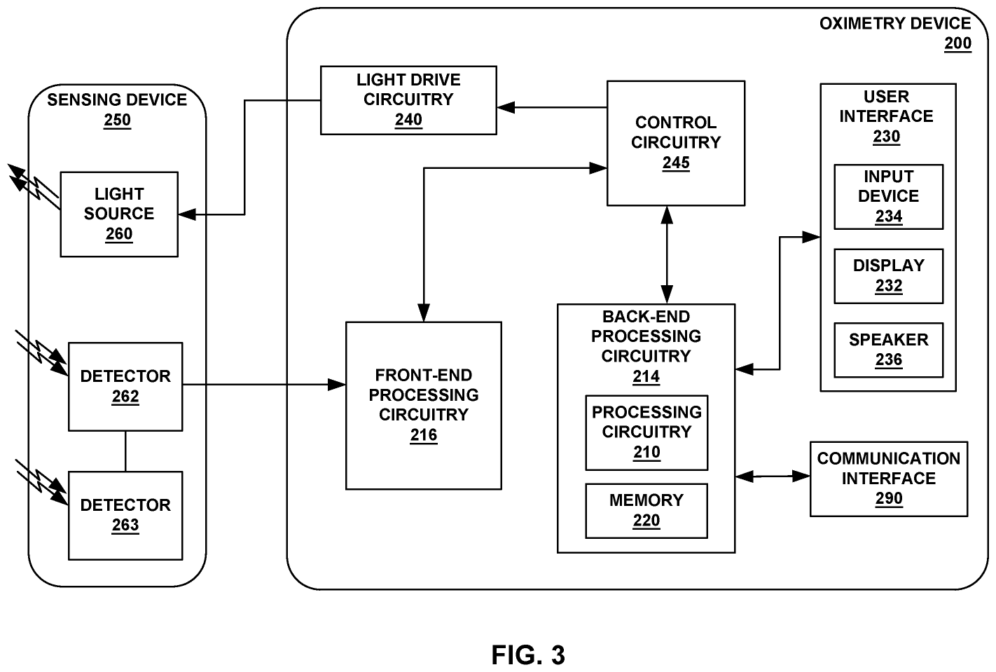

FIG. 3 is a conceptual block diagram illustrating an example oximetry device 200 configured to monitor the heart rate of a patient. While the example of FIG. 3 describes an oximetry device, techniques described herein for determining a transient deceleration or HRV values may be used in other devices, such as, for example, an electrocardiogram monitor or an arterial blood pressure monitor.

In the example shown in FIG. 3, oximetry device 200 is coupled to sensing device 250 and may be collectively referred to as an oximetry system, which each generate and process physiological signals of a subject. Oximetry device 200 and sensing device 250 may be examples of oximetry device 100 and sensing device 150, respectively, of FIG. 1. In some examples, sensing device 250 and oximetry device 200 may be part of an oximeter. As shown in FIG. 3, oximetry device 200 includes back-end processing circuitry 214, user interface 230, light drive circuitry 240, front-end processing circuitry 216, control circuitry 245, and communication interface 290. Oximetry device 200 may be communicatively coupled to sensing device 250. In some examples, oximetry device 200 may also include a blood pressure sensor and/or a blood volume sensor (e.g., sensing devices 151 and 152 of FIG. 1).

In the example shown in FIG. 3, sensing device 250 includes light source 260, detector 262, and detector 263. Light source 260 may be an example of a light source of emitter 160 of FIG. 2. Detectors 262 and 263 may be examples of detector 162 of FIG. 1. In some examples, sensing device 250 may include one detector or more than two detectors. Light source 260 may be configured to emit photonic signals having two or more wavelengths (e.g., up to four or more wavelengths, more than 4 wavelengths, etc.) of light (e.g., red and infrared (IR), or another wavelength of light) into a subject's tissue. For example, light source 260 may include a red light emitting light source and an IR light emitting light source, (e.g., red and IR LEDs), for emitting light into the tissue of a subject to generate physiological signals. In some examples, the red wavelength may be between about 600 nm and about 700 nm, and the IR wavelength may be between about 800 nm and about 1000 nm. Other wavelengths of light may be used in other examples. Light source 260 may include any number of light sources with any suitable characteristics. In examples in which an array of sensors is used in place of sensing device 250, each sensing device may be configured to emit a single wavelength. For example, a first sensing device may emit only a red light while a second sensing device may emit only an IR light. In some examples, light source 260 may be configured to emit two or more wavelengths of near-infrared light (e.g., wavelengths between 600 nm and 1000 nm) into a subject's tissue. In some examples, light source 260 may be configured to emit four wavelengths of light (e.g., 724 nm, 770 nm, 810 nm, and 850 nm) into a subject's tissue. In some examples, the subject may be a medical patient.

As used herein, the term “light” may refer to energy produced by radiative sources and may include one or more of ultrasound, radio, microwave, millimeter wave, infrared, visible, ultraviolet, gamma ray or X-ray electromagnetic radiation. Light may also include any wavelength within the radio, microwave, infrared, visible, ultraviolet, or X-ray spectra, and that any suitable wavelength of electromagnetic radiation may be appropriate for use with the present techniques. Detectors 262 and 263 may be chosen to be specifically sensitive to the chosen targeted energy spectrum of light source 260.

Detectors 262 and 263 may be configured to detect the intensity of multiple wavelengths of near-infrared light. In some examples, detectors 262 and 263 may be configured to detect the intensity of light at the red and IR wavelengths. In some examples, an array of detectors may be used and each detector in the array may be configured to detect an intensity of a single wavelength. In operation, light may enter detector 262 after passing through the subject's tissue, including skin, bone, and other shallow tissue (e.g., non-cerebral tissue and shallow cerebral tissue). Light may enter detector 263 after passing through the subject's tissue, including skin, bone, other shallow tissue (e.g., non-cerebral tissue and shallow cerebral tissue), and deep tissue (e.g., deep cerebral tissue). Detectors 262 and 263 may convert the intensity of the received light into an electrical signal. The light intensity may be directly related to the absorbance and/or reflectance of light in the tissue. That is, when more light at a certain wavelength is absorbed or reflected, less light of that wavelength is received from the tissue by detectors 262 and 263.

For example, detectors 262 and/or detector 263 may determine a first intensity of a first received photonic signal corresponding to a first output photonic signal (e.g., red light) output using a first light emitting diode of light source 260. More specifically, processing circuitry (e.g., light drive circuitry 240) may be configured to drive the first light emitting diode of light source 260 to output the first output photonic signal towards a subject's tissue and receive, from detector 262 and/or detector 263, the first received photonic signal after the first output photonic signal transmits through the subject's tissue. Similarly, detectors 262 and/or detector 263 may determine a second intensity of a second received photonic signal corresponding to a second output photonic signal (e.g., infrared light) output using the second light emitting diode. More specifically, processing circuitry (e.g., light drive circuitry 240) may be configured to drive a second light emitting diode of light source 260 to output the second output photonic signal towards the subject's tissue and receive, from detector 262 and/or detector 263, the second received photonic signal after the second output photonic signal transmits through the subject's tissue.

After converting the received light to an electrical signal, detectors 262 and 263 may send the detection signals to oximetry device 200, which may process the detection signals and determine physiological parameters (e.g., based on the absorption of the red and IR wavelengths in the subject's tissue at both detectors). For example, oximetry device 200 may determine an oxygen saturation level based on the first intensity of the first received photonic signal and the second intensity of the second received photonic signal. More specifically, processing circuitry 210 may estimate a first wavelength for the first output photonic. For instance, processing circuitry 210 may estimate the first wavelength for the first output photonic signal as equal to a first wavelength stored in memory 220. In some instances, processing circuitry 210 may estimate the first wavelength for the first output photonic signal as equal to a first wavelength stored in memory 220 that corresponds to an estimated operating temperature at light source 260 (e.g., the first light emitting diode).

Similarly, processing circuitry 210 may estimate a second wavelength for the second output photonic signal output stored in memory 220. For instance, processing circuitry 210 may estimate the second wavelength for the second output photonic signal as equal to a second wavelength stored in memory 220. In some instances, processing circuitry 210 may estimate the second wavelength for the second output photonic signal as equal to a second wavelength stored in memory 220 that corresponds to an estimated operating temperature at light source 260 (e.g., the second light emitting diode).

In this example, processing circuitry 210 may determine the oxygen saturation level is based on the first wavelength for the first output photonic signal and the second wavelength for the second output photonic signal. For instance, processing circuitry 210 may determine the oxygen saturation level by matching an amount of absorption of the first wavelength (e.g., a difference in magnitude between an emitted light and a received light) and matching an amount of absorption of the second wavelength in a table and outputting a corresponding oxygen saturation level for the absorption of the first wavelength and the absorption of the second wavelength.

Processing circuitry 210 may output an indication of the oxygen saturation level. For example, processing circuitry 210 may store an indication of the oxygen saturation level (e.g., a numerical value indicating the oxygen saturation level) for storage at memory 220. Processing circuitry 210 may output an indication of the oxygen saturation level (e.g., a numerical value indicating the oxygen saturation level) to user interface 230 for output on display 232. Processing circuitry 210 may output an indication of the oxygen saturation level (e.g., a numerical value indicating the oxygen saturation level) to communication interface 290 for storage and/or output at one or more external or implanted devices.

Control circuitry 245 may be coupled to light drive circuitry 240, front-end processing circuitry 216, and back-end processing circuitry 214, and may be configured to control the operation of these components. In some examples, control circuitry 245 may be configured to provide timing control signals to coordinate their operation. For example, light drive circuitry 240 may generate one or more light drive signals, which may be used to turn on and off light source 260, based on the timing control signals provided by control circuitry 245. Front-end processing circuitry 216 may use the timing control signals to operate synchronously with light drive circuitry 240. For example, front-end processing circuitry 216 may synchronize the operation of an analog-to-digital converter and a demultiplexer with the light drive signal based on the timing control signals. In addition, the back-end processing circuitry 214 may use the timing control signals to coordinate its operation with front-end processing circuitry 216.

Light drive circuitry 240, as discussed above, may be configured to generate a light drive signal that is provided to light source 260 of sensing device 250. The light drive signal may, for example, control the intensity of light source 260 and the timing of when light source 260 is turned on and off. In some examples, light drive circuitry 240 provides one or more light drive signals to light source 260. Where light source 260 is configured to emit two or more wavelengths of light, the light drive signal may be configured to control the operation of each wavelength of light. The light drive signal may comprise a single signal or may comprise multiple signals (e.g., one signal for each wavelength of light).

Front-end processing circuitry 216 may perform any suitable analog conditioning of the detector signals. The conditioning performed may include any type of filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof. In some examples, front-end processing circuitry may determine a baseline pulse rate by low pass filtering a sensed pulse rate signal. The conditioned analog signals may be processed by an analog-to-digital converter of front-end processing circuitry 216, which may convert the conditioned analog signals into digital signals. Front-end processing circuitry 216 may operate on the analog or digital form of the detector signals to separate out different components of the signals. Front-end processing circuitry 216 may also perform any suitable digital conditioning of the detector signals, such as low pass, high pass, band pass, notch, averaging, or any other suitable filtering, amplifying, performing an operation on the signal, performing any other suitable digital conditioning, or any combination thereof. Front-end processing circuitry 216 may decrease the number of samples in the digital detector signals. In some examples, front-end processing circuitry 216 may also remove dark or ambient contributions to the received signal.

Back-end processing circuitry 214 may include processing circuitry 210 and memory 220. Processing circuitry 210 may include an assembly of analog or digital electronic components and may be configured to execute software, which may include an operating system and one or more applications, as part of performing the functions described herein with respect to, e.g., processing circuitry 110 of FIG. 1. Processing circuitry 210 may receive and further process physiological signals received from front-end processing circuitry 216. For example, processing circuitry 210 may determine one or more physiological parameter values based on the received physiological signals. For example, processing circuitry 210 may compute one or more of HRV values, blood oxygen saturation (e.g., arterial, venous, or both), pulse rate, respiration rate, respiration effort, blood pressure, hemoglobin concentration (e.g., oxygenated, deoxygenated, and/or total), any other suitable physiological parameters, or any combination thereof.

Processing circuitry 210 may perform any suitable signal processing of a signal, such as any suitable band-pass filtering, adaptive filtering, closed-loop filtering, any other suitable filtering, and/or any combination thereof. Processing circuitry 210 may also receive input signals from additional sources not shown. For example, processing circuitry 210 may receive an input signal containing information about treatments provided to the subject from user interface 230. Additional input signals may be used by processing circuitry 210 in any of the determinations or operations it performs in accordance with back-end processing circuitry 214 or oximetry device 200.

Processing circuitry 210 is an example of processing circuitry 110 and is configured to perform the techniques of this disclosure. For example, processing circuitry 210 may be configured to determine a transient deceleration in the sensed pulse rate signal, remove the transient deceleration from the sensed pulse rate signal to create a modified pulse rate signal, determine an HRV value based on the modified pulse rate signal, and output information indicative of the determined HRV value to user interface 230. In some examples, processing circuitry 210 may also determine a second HRV value based on the sensed pulse rate signal. In such examples, the second HRV value is at least partially based on the transient deceleration, as the transient deceleration still exists in the sensed pulse rate signal. Processing circuitry 210 may output information indicative of the second determined HRV value, for example, to user interface 230. In this manner, a clinician may be able to see the effect the transient deceleration has on HRV values.

Memory 220 may include any suitable computer-readable media capable of storing information that can be interpreted by processing circuitry 210. In some examples, memory 220 may store reference absorption curves, reference sets, determined values, such as blood oxygen saturation, HRV, pulse rate, blood pressure, fiducial point locations or characteristics, initialization parameters, any other determined values, or any combination thereof, in a memory device for later retrieval. Memory 220 may also store thresholds for detecting abrupt changes in blood pressure, and so on. Back-end processing circuitry 214 may be communicatively coupled with user interface 230 and communication interface 290.

User interface 230 may include input device 234, display 232, and speaker 236 in some examples. User interface 230 is an example of user interface 130 shown in FIG. 1, and display 232 is an example of display 132 shown in FIG. 1. User interface 230 may include, for example, any suitable device such as one or more medical devices (e.g., a medical monitor that displays various physiological parameters, a medical alarm, or any other suitable medical device that either displays physiological parameters or uses the output of back-end processing circuitry 214 as an input), one or more display devices (e.g., monitor, personal digital assistant (PDA), mobile phone, tablet computer, clinician workstation, any other suitable display device, or any combination thereof), one or more audio devices, one or more memory devices, one or more printing devices, any other suitable output device, or any combination thereof.

Input device 234 may include one or more of any type of user input device such as a keyboard, a mouse, a touch screen, buttons, switches, a microphone, a joystick, a touch pad, or any other suitable input device or combination of input devices. In other examples, input device 234 may be a pressure-sensitive or presence-sensitive display that is included as part of display 232. Input device 234 may also receive inputs to select a model number of sensing device 250 or blood pressure processing equipment. In some examples, processing circuitry 210 may determine the type of presentation for display 232 based on user inputs received by input device 234.

In some examples, the subject may be a medical patient and display 232 may exhibit a list of values which may generally apply to the subject, such as, for example, an HRV indicator, a pulse rate indicator, an oxygen saturation signal indicator, a blood pressure signal indicator, a COx signal indicator, and/or a COx value indicator. Display 232 may also be configured to present additional physiological parameter information. In some examples, user interface 230 includes speaker 236 that is configured to generate and provide an audible sound that may be used in various examples, such as for example, sounding an audible notification in the event that a physiological parameters of a patient are not within a normal range and/or in the event that processing circuitry 210 determines that sensed blood pressure values may be inaccurate due to a non-physiological reason such as due to movement of a blood pressure probe of blood pressure sensor device 151 (FIG. 1).

In some examples, processing circuitry 210 may determine that a sensed pulse rate of one or more sensed pulse rates associated with a transient deceleration of the one or more transient decelerations falls below a predetermined key threshold and, based on the determination that the sensed pulse rate of the transient deceleration falls below the predetermined key threshold, output information indicative of the transient deceleration being a key or important transient deceleration, for example to display 232 and/or speaker 236. In some examples, display speaker 236 may sound an audible notification of a transient deceleration being a key or important transient deceleration.

Communication interface 290 may enable oximetry device 200 to exchange information with other external or implanted devices. Communication interface 290 may include any suitable hardware, software, or both, which may allow oximetry device 200 to communicate with electronic circuitry, a device, a network, a server or other workstations, a display, or any combination thereof. For example, oximetry device 200 may receive MAP (or other measured blood pressure) values and/or oxygen saturation values from an external device via communication interface 290.

The components of oximetry device 200 that are shown and described as separate components are shown and described as such for illustrative purposes only. In some examples the functionality of some of the components may be combined in a single component. For example, the functionality of front-end processing circuitry 216 and back-end processing circuitry 214 may be combined in a single processor system. Additionally, in some examples the functionality of some of the components of oximetry device 200 shown and described herein may be divided over multiple components. For example, some or all of the functionality of control circuitry 245 may be performed in front-end processing circuitry 216, in back-end processing circuitry 214, or both. In other examples, the functionality of one or more of the components may be performed in a different order or may not be required. In some examples, all of the components of oximetry device 200 can be realized in processing circuitry.

In the above examples, processing circuitry 210 is described as performing the example techniques, wherein front-end processing circuitry 216 may be part of processing circuitry 210. However, any one or combination of processing circuitry 210 and front-end processing circuitry 216 may be configured to perform the example techniques. For instance, the example techniques may be performed by circuitry, and example of the circuitry includes any one or any combination of processing circuitry 210 and front-end processing circuitry 216.

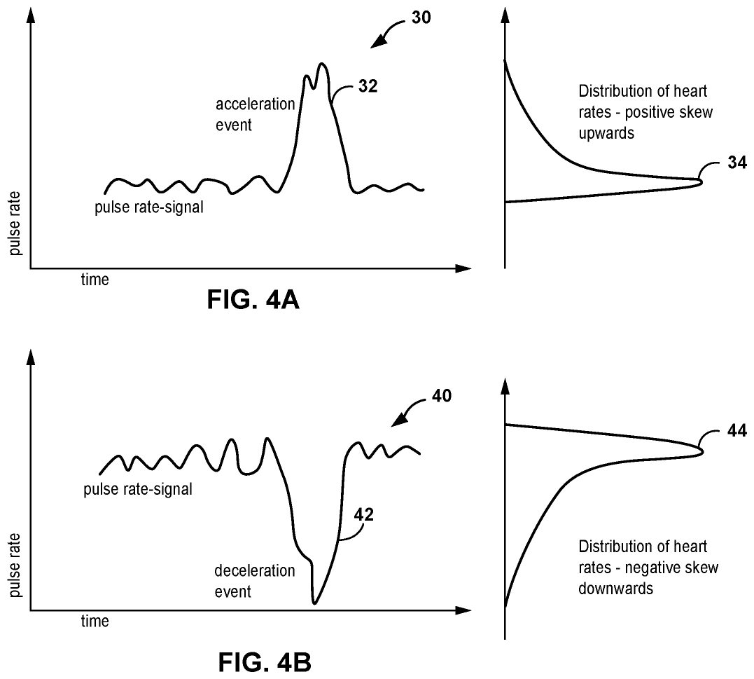

FIGS. 4A and 4B are graphical diagrams illustrating example changes in pulse rate and associated skews to HRV. For example, in FIG. 4A, a pulse rate acceleration 32 is shown in waveform 30 which may result in an associated positive skew 34 in HRV. In FIG. 4B, a pulse rate deceleration 42 is shown in waveform 40 which may result in an associated negative skew 44. Oximetry device 100 may display a distribution to a clinician, facilitating the clinician to be better able to interpret the HRV by taking account the skew (e.g., positive skew 34 and/or negative skew 44) and the possible association between the skew and transient decelerations.

In some examples, oximetry device 100 may determine a deceleration skew associated 44 with the transient deceleration and output information indicative of deceleration skew 44. In some examples, oximetry device 100 may determine one or more accelerations (e.g., acceleration 32) in the sensed pulse rate signal, determine at least one acceleration skew (e.g., acceleration skew 34) associated with at least one of the one or more accelerations, and output information indicative of the at least one acceleration skew.

For example, oximetry device 100 may display skew plots via display 132 and/or an external display over time as a sequence of histograms each representing a specific period, e.g., every 3 hours for the last 24 hours.

In some examples, oximetry device 100 may count accelerations, as well as decelerations. Oximetry device 100 may display a count of both accelerations and decelerations via display 132 or an external display as a way to provide information regarding the skewness to the pulse rate distribution over a defined time period.

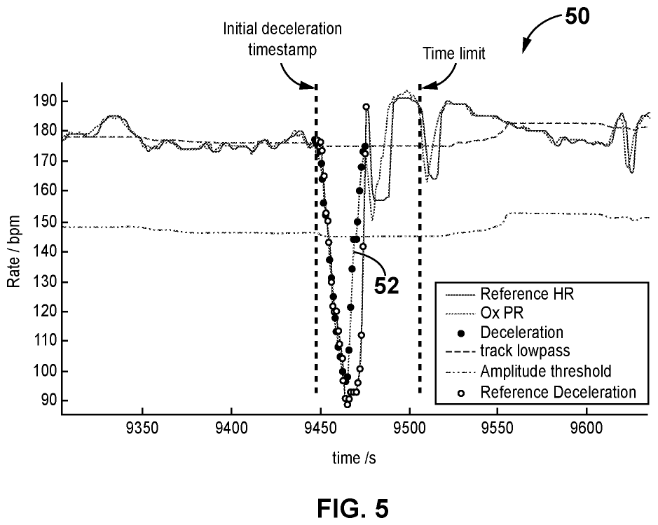

FIG. 5 is a graphical diagram depicting an example classification of a transient deceleration using thresholds. Waveform 50 includes transient deceleration 52. Oximetry device 100 may determine that transient deceleration 52 is a suspected transient deceleration. For example, oximetry device 100 may determine that transient deceleration 52 is a rapid deceleration that more or less returns to a baseline pulse rate. As an example of a matching criteria, in some examples, oximetry device 100 may confirm a suspected occurrence to be an actual transient deceleration if the suspected occurrence meets the following: 1) Find (Deceleration_initial_time) by checking if a rate derivative is positive going backwards, which may include applying a smoothing filter to the deceleration profile; 2) Amplitude>amplitude_threshold [e.g., 30 bpm]; 3) Deceleration_amplitude>constant*deceleration_period, e.g., constant=1 bpm/s; and 4) Period<=time_limit [e.g., 120 seconds]. In some examples, oximetry device 100 may remove transient deceleration 52 from the sensed pulse rate signal (e.g., waveform 50) to create a modified pulse rate signal. In some examples, oximetry device 100 may determine a HRV based on the modified pulse rate signal.

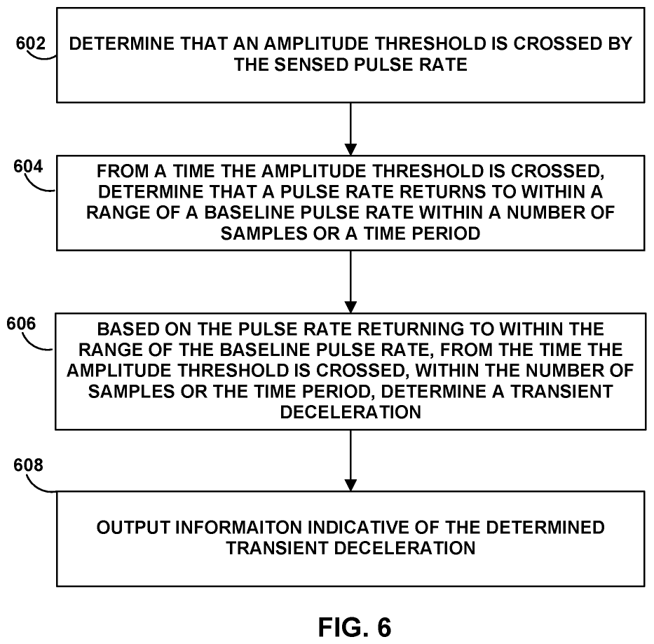

FIG. 6 is a flow diagram illustrating an example technique for determining a transient deceleration. Although FIG. 6 is described with respect to oximetry device 100 (FIG. 2), in other examples, other devices may perform any part of the technique of FIG. 6.

Oximetry device 100 may determine that an amplitude threshold is crossed by the sensed pulse rate signal (602). For example, oximetry device 100 may compare an amplitude threshold to an amplitude of the sensed pulse rate signal and determine that the sensed pulse rate signal falls below the amplitude threshold.

Oximetry device 100 may, from a time the amplitude threshold is crossed by the sensed pulse rate signal, determine that a pulse rate returns to within a range of a baseline pulse rate within a number of samples or a time period (604). For example, oximetry device 100 may compare a pulse rate of the sensed pulse rate signal after the sensed pulse rate signal crosses the amplitude threshold to a baseline pulse rate to determine whether the pulse rate returns to within the range of the baseline pulse rate.

Oximetry device 100 may, based on the pulse rate returning to within the predetermined range of the baseline pulse rate, from the time the amplitude threshold is crossed, within the predetermined number of samples or the predetermined time period, determine a transient deceleration (606). For example, oximetry device 100 may classify the portion of the sensed pulse rate signal from when the sensed pulse rate signal crosses the amplitude threshold to when the pulse rate returns to within the predetermined range of the baseline pulse rate as a transient deceleration.

Oximetry device 100 may output information indicative of the determined transient deceleration (608). For example, oximetry device 100 may output an indication of the determined transient deceleration, such as a label, graph, or the like, to display 132.

In some examples, oximetry device 100 may remove one or more transient decelerations from the sensed pulse rate signal to create a modified pulse rate signal. Oximetry device 100 may determine an HRV value based on the modified pulse rate signal and output information indicative of the determined HRV value.

In some examples, the HRV value is a first HRV value and the determined HRV value is a first determined HRV value, and oximetry device 100 may determine a second HRV value based on the sensed pulse rate signal, wherein the second HRV value is at least partially based on the one or more transient decelerations, and output information indicative of the second determined HRV value.

In some examples, oximetry device 100 may determine a count of one or more transient decelerations and output information indicative of the count of the one or more transient decelerations. In some examples, oximetry device 100 may associate each of the one or more transient decelerations with a respective timestamp, wherein the count is sortable according to a user selectable time period based on respective timestamps. In some examples, oximetry device 100 may determine a blood oxygen saturation (SpO2) value coinciding with each of the one or more transient decelerations, wherein the count is sortable according to the SpO2 value, according to when the SpO2 value drops below a predetermined threshold, or according to an SpO2 value drop. In some examples, wherein the count is sortable according to a period and amplitude of a sensed pulse rate of each of the one or more transient decelerations.

In some examples, oximetry device 100 may determine that a sensed pulse rate of one or more sensed pulse rates associated with a transient deceleration of the one or more transient decelerations falls below a predetermined key threshold and, based on the determination that the sensed pulse rate of the transient deceleration falls below the predetermined key threshold, output information indicative of the transient deceleration being a key transient deceleration.

In some examples, oximetry device 100 may determine at least one deceleration skew associated with at least one of the one or more transient decelerations and output information indicative of the at least one deceleration skew.

In some examples, oximetry device 100 may determine one or more accelerations in the sensed pulse rate signal. Oximetry device 100 may determine at least one acceleration skew associated with at least one of the one or more accelerations and output information indicative of the at least one acceleration skew.

In some examples, oximetry device 100 may determine the baseline pulse rate based on the sensed pulse rate signal. For example, oximetry device 100 may use low pass filtering or tracking to determine a baseline pulse rate.

In some examples, to determine the transient deceleration oximetry device 100 may determine one or more of the following: a) that an amplitude of the sensed pulse rate signal between an initial timestamp associated with a suspected transient deceleration and a minimum value of the pulse rate signal associated with the suspected transient deceleration is greater than or equal to the amplitude threshold, b) that a period of a portion of the sensed pulse rate signal coinciding with the suspected transient deceleration is within a predetermined time limit, c) that an amplitude of a return of the sensed pulse rate signal from the minimum value of the sensed pulse rate signal associated with the suspected transient deceleration is within a ratio (e.g., a predetermined ratio) of the amplitude from the initial timestamp associated with the suspected transient deceleration to the minimum value, or d) that a ratio between the period and an amplitude of the portion of the sensed pulse rate signal coinciding with the suspected transient deceleration are less than a ratio threshold (e.g., a predetermined ratio threshold). Oximetry device 100 may, based on the determining of the one or more of the following, classify the suspected transient deceleration as a transient deceleration.

The disclosure contemplates computer-readable storage media comprising instructions to cause a processor to perform any of the functions and techniques described herein. The computer-readable storage media may take the example form of any volatile, non-volatile, magnetic, optical, or electrical media, such as a RAM, ROM, NVRAM, EEPROM, or flash memory. The computer-readable storage media may be referred to as non-transitory. A programmer, such as patient programmer or clinician programmer, or other computing device may also contain a more portable removable memory type to enable easy data transfer or offline data analysis.

The techniques described in this disclosure, including those attributed to devices 100 and 200, processing circuitry 110, 210, 214, and 216, memories 120 and 220, displays 132 and 232, sensing circuitries 140-142, circuitries 240 and 245, sensing devices 150, 151, 152, and 250, and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in patient monitors, such as multiparameter patient monitors (MPMs) or other devices, remote servers, or other devices. The term “processor” or “processing circuitry” may generally refer to any of the foregoing logic circuitry, alone or in combination with other logic circuitry, or any other equivalent circuitry.

As used herein, the term “circuitry” refers to an ASIC, an electronic circuit, a processor (shared, dedicated, or group) and memory that execute one or more software or firmware programs, a combinational logic circuit, or other suitable components that provide the described functionality. The term “processing circuitry” refers one or more processors distributed across one or more devices. For example, “processing circuitry” can include a single processor or multiple processors on a device. “Processing circuitry” can also include processors on multiple devices, wherein the operations described herein may be distributed across the processors and devices.

Such hardware, software, firmware may be implemented within the same device or within separate devices to support the various operations and functions described in this disclosure. For example, any of the techniques or processes described herein may be performed within one device or at least partially distributed amongst two or more devices, such as between devices 100 and 200, processing circuitry 110, 210, 214, and 216, memories 120 and 220, sensing circuitries 140-142, and/or circuitries 240 and 245. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may also be embodied or encoded in an article of manufacture including a non-transitory computer-readable storage medium encoded with instructions. Instructions embedded or encoded in an article of manufacture including a non-transitory computer-readable storage medium encoded, may cause one or more programmable processors, or other processors, to implement one or more of the techniques described herein, such as when instructions included or encoded in the non-transitory computer-readable storage medium are executed by the one or more processors. Example non-transitory computer-readable storage media may include RAM, ROM, programmable ROM (PROM), erasable programmable ROM (EPROM), electronically erasable programmable ROM (EEPROM), flash memory, a hard disk, a compact disc ROM (CD-ROM), a floppy disk, a cassette, magnetic media, optical media, or any other computer readable storage devices or tangible computer readable media.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache). Elements of devices and circuitry described herein, including, but not limited to, devices 100 and 200, processing circuitry 110, 210, 214, and 216, memories 120 and 220, displays 132 and 232, sensing circuitries 140-142, circuitries 240 and 245, sensing devices 150-152 and 250 may be programmed with various forms of software. The one or more processors may be implemented at least in part as, or include, one or more executable applications, application modules, libraries, classes, methods, objects, routines, subroutines, firmware, and/or embedded code, for example.

This disclosure includes the following non-limiting examples.

Example 1. A device for determining a transient deceleration, the device comprising: a memory configured to store a sensed pulse rate signal indicative of one or more sensed pulse rates; and processing circuitry configured to: determine that an amplitude threshold is crossed by the sensed pulse rate signal; from a time the amplitude threshold is crossed, determine that a pulse rate returns to within a range of a baseline pulse rate within a number of samples or a time period; based on the pulse rate returning to within the range of the baseline pulse rate, from the time the amplitude threshold is crossed, within the number of samples or the time period, determine a transient deceleration; and output information indicative of the determined transient deceleration.

Example 2. The device of example 1, wherein the processing circuitry is further configured to: remove the transient deceleration from the sensed pulse rate signal to create a modified pulse rate signal; determine a heart rate variability (HRV) value based on the modified pulse rate signal; and output information indicative of the determined HRV value.

Example 3. The device of example 2, wherein the HRV value is a first HRV value and the determined HRV value is a first determined HRV value, wherein the processing circuitry is further configured to: determine a second HRV value based on the sensed pulse rate signal, wherein the second HRV value is at least partially based on the transient deceleration; and output information indicative of the second determined HRV value.

Example 4. The device of any of examples 1-3, wherein the processing circuitry is further configured to: determine a count of one or more transient decelerations; and output information indicative of the count of the one or more transient decelerations.

Example 5. The device of example 4, wherein the processing circuitry is further configured to: associate each of the one or more transient decelerations with a respective timestamp, wherein the count is sortable according to a user selectable time period based on respective timestamps.

Example 6. The device of example 4 or example 5, wherein the processing circuitry is further configured to: determine a blood oxygen saturation (SpO2) value coinciding with each of the one or more transient decelerations, wherein the count is sortable according to the SpO2 value, according to when the SpO2 value drops below a predetermined threshold, or according to an SpO2 value drop.

Example 7. The device of any of examples 4-6, wherein the count is sortable according to a period and amplitude of a sensed pulse rate of each of the one or more transient decelerations.

Example 8. The device of any of examples 1-7 wherein the processing circuitry is further configured to: determine that a sensed pulse rate of one or more sensed pulse rates associated with the transient deceleration falls below a predetermined key threshold; and based on the determination that the sensed pulse rate of the transient deceleration falls below the predetermined key threshold, output information indicative of the transient deceleration being a key transient deceleration.

Example 9. The device of any of examples 1-8, wherein the processing circuitry is further configured to: determine a deceleration skew associated with the transient deceleration; and output information indicative of the deceleration skew.

Example 10. The device of any of examples 1-9, wherein the processing circuitry is further configured to: determine one or more accelerations in the sensed pulse rate signal; determine at least one acceleration skew associated with at least one of the one or more accelerations; and output information indicative of the at least one acceleration skew.

Example 11. The device of any of examples 1-10, wherein the processing circuitry is further configured to determine the baseline pulse rate based on the sensed pulse rate signal.

Example 12. The device of any of examples 1-11, wherein to determine the transient deceleration the processing circuitry is configured to: determine one or more of the following: a) that an amplitude of the sensed pulse rate signal between an initial timestamp associated with a suspected transient deceleration and a minimum value of the pulse rate signal associated with the suspected transient deceleration is greater than or equal to the amplitude threshold, b) that a period of a portion of the sensed pulse rate signal coinciding with the suspected transient deceleration is within a predetermined time limit, c) that an amplitude of a return of the sensed pulse rate signal from the minimum value of the sensed pulse rate signal associated with the suspected transient deceleration is within a ratio of the amplitude from the initial timestamp associated with the suspected transient deceleration to the minimum value, or d) that a ratio between the period and an amplitude of the portion of the sensed pulse rate signal coinciding with the suspected transient deceleration are less than a ratio threshold; and based on the determining of the one or more of the following, classify the suspected transient deceleration as a transient deceleration.

Example 13. A method for determining a transient deceleration, the method comprising: determining, by processing circuitry, that an amplitude threshold is crossed by a sensed pulse rate signal indicative of one or more sensed pulse rates; from a time the amplitude threshold is crossed, determining, by the processing circuitry, that a pulse rate returns to within a range of a baseline pulse rate within a number of samples or a time period; based on the pulse rate returning to within the range of the baseline pulse rate, from the time the amplitude threshold is crossed, within the number of samples or the time period, determining, by the processing circuitry, a transient deceleration; and outputting, by the processing circuitry, information indicative of the determined transient deceleration.

Example 14. The method of example 13, further comprising: removing, by the processing circuitry, the transient deceleration from the sensed pulse rate signal to create a modified pulse rate signal; determining, by the processing circuitry, an HRV value based on the modified pulse rate signal; and outputting, by the processing circuitry, information indicative of the determined HRV value.

Example 15. The method of example 14, wherein the HRV value is a first HRV value and the determined HRV value is a first determined HRV value, further comprising: determining, by the processing circuitry, a second HRV value based on the sensed pulse rate signal, wherein the second HRV value is at least partially based on the transient deceleration; and outputting, by the processing circuitry, information indicative of the second determined HRV value.

Example 16. The method of any of examples 13-15, further comprising: determining, by the processing circuitry, a count of one or more transient decelerations; and outputting, by the processing circuitry, information indicative of the count of the one or more transient decelerations.

Example 17. The method of example 16, further comprising: associating, by the processing circuitry, each of the one or more transient decelerations with a respective timestamp, wherein the count is sortable according to a user selectable time period based on respective timestamps.

Example 18. The method of example 16 or example 17, further comprising: determining, by the processing circuitry, a blood oxygen saturation (SpO2) value coinciding with each of the one or more transient decelerations, wherein the count is sortable according to the SpO2 value, according to when the SpO2 value drops below a predetermined threshold, or according to an SpO2 value drop.

Example 19. The method of any of examples 16-18, wherein the count is sortable according to a period and amplitude of a sensed pulse rate of each of the one or more transient decelerations.

Example 20. The method of any of examples 13-19, further comprising determining, by the processing circuitry, that a sensed pulse rate of one or more sensed pulse rates associated with the transient deceleration falls below a predetermined key threshold; and based on the determination that the sensed pulse rate of the transient deceleration falls below the predetermined key threshold, outputting, by the processing circuitry, information indicative of the transient deceleration being a key transient deceleration.

Example 21. The method of any of examples 13-20, further comprising: determining, by the processing circuitry, a deceleration skew associated with the transient deceleration; and outputting, by the processing circuitry, information indicative of the deceleration skew.

Example 22. The method of any of examples 13-21, further comprising: determining, by the processing circuitry, one or more accelerations in the sensed pulse rate signal; determining, by the processing circuitry, at least one acceleration skew associated with at least one of the one or more accelerations; and outputting, by the processing circuitry, information indicative of the at least one acceleration skew.

Example 23. The method of any of examples 13-22, further comprising: determining, by the processing circuitry, the baseline pulse rate based on the sensed pulse rate signal.

Example 24. The method of any of examples 13-23, wherein determining the transient deceleration comprises determining one or more of the following: a) that an amplitude of the sensed pulse rate signal between an initial timestamp associated with a suspected transient deceleration and a minimum value of the pulse rate signal associated with the suspected transient deceleration is greater than or equal to the amplitude threshold, b) that a period of a portion of the sensed pulse rate signal coinciding with the suspected transient deceleration is within a predetermined time limit, c) that an amplitude of a return of the sensed pulse rate signal from the minimum value of the sensed pulse rate signal associated with the suspected transient deceleration is within a ratio of the amplitude from the initial timestamp associated with the suspected transient deceleration to the minimum value, or d) that a ratio between the period and an amplitude of the portion of the sensed pulse rate signal coinciding with the suspected transient deceleration are less than a ratio threshold; and based on the determining of the one or more of the following, classify the suspected transient deceleration as a transient deceleration.

Example 25. A non-transitory computer-readable storage medium storing instructions, which, when executed cause processing circuitry to: determine that an amplitude threshold is crossed by a sensed pulse rate signal indicative of one or more sensed pulse rates; from a time the amplitude threshold is crossed determine that a pulse rate returns to within a range of a baseline pulse rate within a number of samples or a time period; based on the pulse rate returning to within the range of the baseline pulse rate, from the time the amplitude threshold is crossed, within the number of samples or the time period, determine a transient deceleration; and output information indicative of the determined transient deceleration.

Various examples of the disclosure have been described. Any combination of the described systems, operations, or functions is contemplated. These and other examples are within the scope of the following claims.

What is claimed is:

1. A device for determining a transient deceleration, the device comprising:
a memory configured to store a sensed pulse rate signal indicative of one or more sensed pulse rates; and
processing circuitry configured to:
determine that an amplitude threshold is crossed by the sensed pulse rate signal;
from a time the amplitude threshold is crossed, determine that a pulse rate returns to within a range of a baseline pulse rate within a number of samples or a time period;
based on the pulse rate returning to within the range of the baseline pulse rate, from the time the amplitude threshold is crossed, within the number of samples or the time period, determine a transient deceleration; and
output information indicative of the determined transient deceleration.

2. The device of claim 1, wherein the processing circuitry is further configured to:
remove the transient deceleration from the sensed pulse rate signal to create a modified pulse rate signal;
determine a heart rate variability (HRV) value based on the modified pulse rate signal; and
output information indicative of the determined HRV value.

3. The device of claim 2, wherein the HRV value is a first HRV value and the determined HRV value is a first determined HRV value, wherein the processing circuitry is further configured to:
determine a second HRV value based on the sensed pulse rate signal, wherein the second HRV value is at least partially based on the transient deceleration; and
output information indicative of the second determined HRV value.

4. The device of claim 1, wherein the processing circuitry is further configured to:
determine a count of one or more transient decelerations; and
output information indicative of the count of the one or more transient decelerations.

5. The device of claim 4, wherein the processing circuitry is further configured to:
associate each of the one or more transient decelerations with a respective timestamp,
wherein the count is sortable according to a user selectable time period based on respective timestamps.

6. The device of claim 4, wherein the processing circuitry is further configured to:
determine a blood oxygen saturation (SpO2) value coinciding with each of the one or more transient decelerations,
wherein the count is sortable according to the SpO2 value, according to when the SpO2 value drops below a predetermined threshold, or according to an SpO2 value drop.

7. The device of claim 4, wherein the count is sortable according to a period and amplitude of a sensed pulse rate of each of the one or more transient decelerations.

8. The device of claim 1, wherein the processing circuitry is further configured to:
determine that a sensed pulse rate of one or more sensed pulse rates associated with the transient deceleration falls below a predetermined key threshold; and
based on the determination that the sensed pulse rate of the transient deceleration falls below the predetermined key threshold, output information indicative of the transient deceleration being a key transient deceleration.

9. The device of claim 1, wherein the processing circuitry is further configured to:
determine a deceleration skew associated with the transient deceleration; and
output information indicative of the deceleration skew.

10. The device of claim 1, wherein the processing circuitry is further configured to:
determine one or more accelerations in the sensed pulse rate signal;
determine at least one acceleration skew associated with at least one of the one or more accelerations; and
output information indicative of the at least one acceleration skew.

11. The device of claim 1, wherein the processing circuitry is further configured to determine the baseline pulse rate based on the sensed pulse rate signal.

12. The device of claim 1, wherein to determine the transient deceleration the processing circuitry is configured to determine one or more of the following:
a) that an amplitude of the sensed pulse rate signal between an initial timestamp associated with a suspected transient deceleration and a minimum value of the pulse rate signal associated with the suspected transient deceleration is greater than or equal to the amplitude threshold,
b) that a period of a portion of the sensed pulse rate signal coinciding with the suspected transient deceleration is within a predetermined time limit,
c) that an amplitude of a return of the sensed pulse rate signal from the minimum value of the sensed pulse rate signal associated with the suspected transient deceleration is within a ratio of the amplitude from the initial timestamp associated with the suspected transient deceleration to the minimum value, or
d) that a ratio between the period and an amplitude of the portion of the sensed pulse rate signal coinciding with the suspected transient deceleration are less than a ratio threshold; and
based on the determining of the one or more of the following, classify the suspected transient deceleration as a transient deceleration.

13. A method for determining a transient deceleration, the method comprising:
determining, by processing circuitry, that an amplitude threshold is crossed by a sensed pulse rate signal indicative of one or more sensed pulse rates;
from a time the amplitude threshold is crossed, determining, by the processing circuitry, that a pulse rate returns to within a range of a baseline pulse rate within a number of samples or a time period;
based on the pulse rate returning to within the range of the baseline pulse rate, from the time the amplitude threshold is crossed, within the number of samples or the time period, determining, by the processing circuitry, a transient deceleration; and
outputting, by the processing circuitry, information indicative of the determined transient deceleration.

14. The method of claim 13, further comprising:
removing, by the processing circuitry, the transient deceleration from the sensed pulse rate signal to create a modified pulse rate signal;
determining, by the processing circuitry, an HRV value based on the modified pulse rate signal; and
outputting, by the processing circuitry, information indicative of the determined HRV value.

15. The method of claim 14, wherein the HRV value is a first HRV value and the determined HRV value is a first determined HRV value, further comprising:
determining, by the processing circuitry, a second HRV value based on the sensed pulse rate signal, wherein the second HRV value is at least partially based on the transient deceleration; and
outputting, by the processing circuitry, information indicative of the second determined HRV value.

16. The method of claim 13, further comprising:
determining, by the processing circuitry, a count of one or more transient decelerations; and
outputting, by the processing circuitry, information indicative of the count of the one or more transient decelerations.

17. The method of claim 16, further comprising:
associating, by the processing circuitry, each of the one or more transient decelerations with a respective timestamp,
wherein the count is sortable according to a user selectable time period based on respective timestamps.

18. The method of claim 16, further comprising:
determining, by the processing circuitry, a blood oxygen saturation (SpO2) value coinciding with each of the one or more transient decelerations,
wherein the count is sortable according to the SpO2 value, according to when the SpO2 value drops below a predetermined threshold, or according to an SpO2 value drop.

19. The method of claim 16, wherein the count is sortable according to a period and amplitude of a sensed pulse rate of each of the one or more transient decelerations.

20. A non-transitory computer-readable storage medium storing instructions, which, when executed cause processing circuitry to:
determine that an amplitude threshold is crossed by a sensed pulse rate signal indicative of one or more sensed pulse rates;
from a time the amplitude threshold is crossed determine that a pulse rate returns to within a range of a baseline pulse rate within a number of samples or a time period;
based on the pulse rate returning to within the range of the baseline pulse rate, from the time the amplitude threshold is crossed, within the number of samples or the time period, determine a transient deceleration; and
output information indicative of the determined transient deceleration.
